(19) United States
(12) Patent Application Publication
(10) Pub. No.: US 2004/0265404 A1
(43) Pub. Date: Dec. 30, 2004

(54) Extract of ceratonia siliqua leaves and pods containing polyphenols with antioxidant and antitumor activities

(76) Inventor: Mario Baraldi, Modena (IT)

Correspondence Address: YOUNG & THOMPSON, 745 SOUTH 23RD STREET, 2ND FLOOR, ARLINGTON, VA, 22202, US

(21) Appl. No.: 10/492,594

(22) PCT Filed: Oct. 10, 2002
(86) PCT No.: PCT/EP02/11356

(30) Foreign Application Priority Data
Oct. 17, 2001 (IT) MI2001A002141

(51) Int. Cl. A61K 35/78

(52) U.S. Cl. 424/774

(57) ABSTRACT

The use of an extract of Ceratonia siliqua leaves and pod containing polyphenols (gallic acid, (−)-epigallocatechin gallate, (−)-epicatechin gallate) with oxidant activity and potential antitumor activity, useful for the preparation of pharmaceutical compositions, dietary supplements or extemporaneous preparations for the alimentary use, as well as processes for the preparation of the extracts and separation and quantitation of gallic acid, (−)-epigallocatechin gallate, (−)-epicatechin gallate.

The present invention relates to an extract of Ceratoizia siliqua leaves and pod containing polyphenols (gallic acid, (−)-epigallocatechin gallate, (−)-epicatechin gallate) with antioxidant activity and potential antitumor activity, useful for the preparation of pharmaceutical compositions, dietary supplements or extemporaneous preparations for the alimentary use. Said extract is characterized by high content in polyphenols as well as absence of central nervous system-stimulating substances, such as caffeine and theophylline.

### TECHNICAL BACKGROUND

According to numerous papers in literature, diet rich in fruit and vegetables apparently concurs in preventing tumors of the gastrointestinal tract [J. M. Pezzetto, Biochein. Pharmacol. 53:121-123, 1997]. Vegetables, in fact, contain a number of compounds with antioxidant and antitumor activities, inter alia flavonoids and polyphenols, which are considered able to interfere with the hepatic microsomal system, preventing it from activating the cancerogenic substances found in food and environment.

Flavonoids are a heterogeneous group of vegetable polyphenols (more than 4000) sharing a benzopyrane structure. They constitute pigments (orange, red and blue colours) of various vegetable (flowers, fruit, leaves) and are important factors for the growth, development and protection of plants. The major flavonoids with antioxidant activity present in vegetables are: (+)-catechin, (−)-epicatechin, (−)-epigallocatechin and the corresponding gallic acid esters: (−)-epicatechin gallate and (−)-epigallocatechin gallate [Blot et al. Am. J. Cancer Prevention 62:1477-82, 1992].

A number of studies reported the antitumor activity of these compounds [Cao Y. and Cao R. Nature 398:-391, 1999], in particular of (−)-epigallocatechin gallate (EGCG). EGCG takes part in:

regulating the cellular cycle, in particular through dose-dependent inhibition of mitosis by blocking cells in the G0-G1 phase;

inducing apoptosis (programmed cell death) in various tumour cells, but not in normal cells. This process is important for maintaining cell growth and proliferation balances. Tumour cells growth is in fact due to escape from the normal control mechanisms, which also act through regulation of apoptosis;

inhibiting urokinase, a proteolytic enzyme which allows tumour cells to invade the adjacent cells and form metastasis;

inhibiting angiogenesis;

eliminating free radicals thanks to its high antioxidant power.

Among the commercially available vegetable products, green and black teas have antioxidant and antitumour activities, due to their high contents in flavonoids; however, not all individuals can tolerate them due to the presence of stimulant substances, such as caffeine and theophylline. Vegetable products containing flavonoids but free from any stimulating substances, for use in the prevention and/or treatment of degenerative diseases and in particular of tumors [Fujilci H. et al. Nutrition Reviews 54: 67-70, 1996] would therefore be valuable.

The present invention relates to an extract, either dry or aqueous, which has high content in polyphenols while being free from stimulating substances, obtained from carob (an evergreen tree with wide leafage, belonging to the order Rosales, family Papillioizacee, subfamily Cesalpinoidee, genus Ceratoniia, species Ceratonia siliqua).

### DETAILED DISCLOSURE OF THE INVENTION

The present invention relates to the use of a dry or aqueous extract of Ceratonia siliqua (carob) leaves and pod, containing polyphenols, for the preparation of pharmaceutical compositions, dietary supplements or extemporaneous preparations for the alimentary use, having antioxidant activity and potential antitumor activity. The term “polyphenols” herein preferably means gallic acid, (−)-epigallocatechin gallate and (−)-epicatechin gallate.

The present invention further relates to the process for the preparation of said extract.

Carob pod leaves or meal infused in hot distilled water for 15 min, preferably in a ratio of 1 g of leaves or meal to 50-200 ml of water, most preferably 1 g of leaves or meal-to 100 ml of water. The aqueous extract is then centrifuged and filtered. A second aqueous extract is optionally obtained by further infusing the centrifugation residue. The aqueous extract or the combined aqueous extracts are evaporated to obtain a dry extract, which consists solely of water-soluble substances and can therefore be easily dissolved and is well-suited for the preparation of formulations such as sachets, syrups, capsules. This extract is free from central nervous system-stimulating substances: as a consequence, it is well tolerated and does not require costly decaffeinization or deteination processes.

According to a preferred embodiment of the invention, the extract is prepared by infusing 1 g of carob leaves or pod meal in 100 ml of hot distilled water for 15 min. The aqueous extract is then centrifuged at 13,000 rpm for 10 min and filtered through PTFE 0.22 μm filters. A second aqueous extract is obtained repeating the procedure on the centrifugation residue.

Carob leaves and pod contain fair amounts of polyphenols, mainly catechins (gallic acid, (−) epicatechin gallate, (−) epigallocatechin gallate), which have been separated and purified by chromatography (HPLC). The obtained amounts are (Table n°1, example 2) 6.28 mg/g dry leaves for the leaves and 1.36 mg/g of meal for the pod meal, respectively.

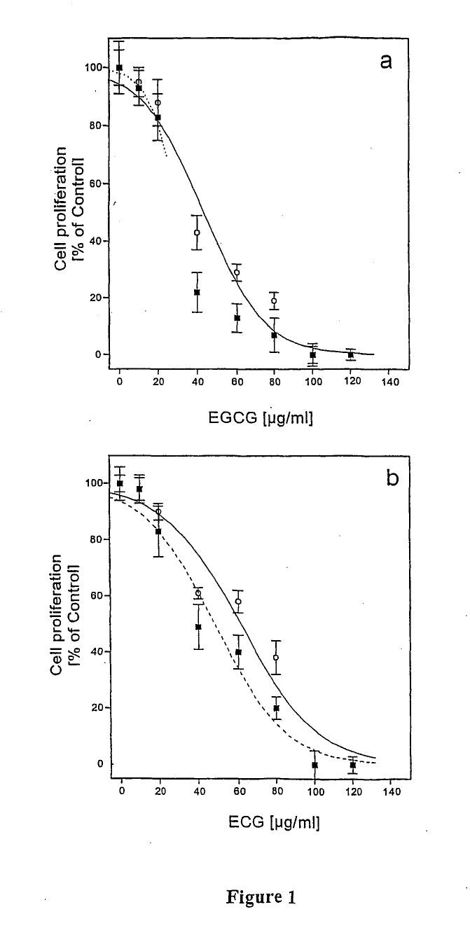

Biological tests carried out using the above mentioned catechins evidenced a marked, dose-dependent reduction in the proliferation of hepatic tumour cells cultures. FIG. 1 shows the dose-dependent activities of EGCG (a) and EGC (b) on the inhibition of cell proliferation in liver tumour cells after 24 (white dots) and 48 hours (black dots) exposure. (−)-Epigallocatechin gallate (EGCG) and (−)-epicatechin gallate (ECG) inhibit the proliferation already at concentrations of 40 μg/ml, reaching the peak activity at concentrations of 80 μg/ml. Furthermore, said reduction turned out to be higher for 48 hours treatments compared with 24 hours treatments.

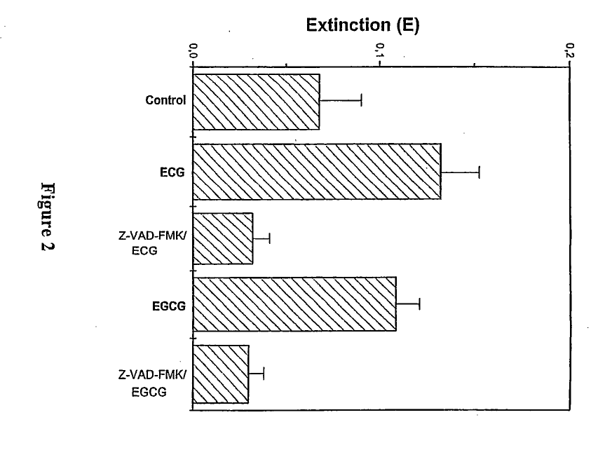

Moreover, catechins at concentrations of 80 μg/ml induce apoptosis causing a significant increase in the caspase 3 protease activity compared with untreated cells. FIG. 2 shows the activation of the protease activity exerted by ECG and EGCG alone and in combination with a caspase 3 specific inhibitor (Z-VAD-FMK) expressed as arbitrary units referred to the optical density of the used instrumentation.

The invention is illustrated in greater detail by the following examples.

### EXAMPLE 1

Preparation of Standards and Samples

Standards consisted of a mixture of (+)-catechin (C), (−)-epicatechin (EC), (−)-epigallocatechin-3-gallate (EGCG), (−)-epicatechin-3-gallate (ECG), gallic acid (GAc), theophylline (T), caffeine (Ca), chlorogenic acid (ClAc), caffeic acid (Cac), catechol (Ct) and tea extracts, containing teaflavin, teaflavin-3 -monogallate, teaflavin-3 ′-monogallate and teaflavin-3,3′-digallate (with purity >80%) were prepared dissolving known amounts of all the analytes in the mobile phase used for the chromatographic separation so as to reach concentrations from 0.3 to 500 gg/ml. Samples were prepared by infusing 1 g of carob leaves or pod meal in 100 ml of hot distilled water for 15 min. Afterwards, the aqueous extract was centrifuged at 13,000 rpm for 10 min, then filtered through PTFE 0.22 μm filters. A second aqueous extract was obtained from the centrifugation residue.

### Example 2

Chromatographic Analysis

The chromatographic separation was carried out with an analytic Microsorb RP-C₁₈ 100 A column (250×4.6 mm), thermostatized at 40° C, using a gradient with starting composition: 68% water, 22% of 0.5% phosphoric acid, 4% acetonitrile and 6% methanol, linearly increasing the organic solvents with a flow of 1 ml/min. UV-Vis absorption spectra (λ=198-708 nm) were recorded continuously and the samples absorption peaks were identified by comparing retention times and UV-Vis spectra with those of the standard. The peak area at 205 nm was used to establish calibration curves.

TABLE 1
Catechins quantitation by chromatographic analysis

| | CAROB LEAVES | | | CAROB MEAL | | |
|---|---|---|---|---|---|---|
| | mg/g ± s.d.* | | | mg/g ± s.d.* | | |
| | 1^{st} infusion | 2^{nd} infusion | Total | 1^{st} infusion | 2^{nd} infusion | Total |
| G Ac | 3.34 ± 0.032 | 0.96 ± 0.020 | 4.30 | 0.87 ± 0.000 | 0.33 ± 0.007 | 1.20 |
| EGC | — | — | — | 0.05 ± 0.000 | 0.01 ± 0.001 | 0.06 |
| C | — | — | — | 0.01 ± 0.000 | — | 0.01 |
| EGCG | 1.23 ± 0.409 | 0.28 ± 0.030 | 1.51 | 0.01 ± 0.000 | — | 0.01 |
| EC | — | — | — | — | — | — |
| ECG | 0.47 ± 0.095 | — | 0.47 | 0.06 ± 0.000 | 0.02 ± 0.004 | 0.08 |
| Total | 5.04 ± 0.395 | 1.24 ± 0.049 | 6.28 | 1.00 ± 0.008 | 0.36 ± 0.002 | 1.36 |

*= Means and Standard Deviations for 3 determinations

### Example 3

Pharmacological Experimentation

Cell cultures

Tumour cell cultures (T1) used in the tests derive from transgenic mice hepatocellular carcinoma, in which an over-expression of the c-myc and TGFαhad been induced. Cells were kept in L-glutamine-free DMEM medium, HAM F 12 containing 1 mg/ml galactose, 18 mM HEPES, 5 mM Na piruvate, 30 mg/Ml proline, ITS 100×, 2 mM glutamine, 0.1% gentamicin (Sigma-Aldrich, Italy) and added with 10% FBS. Cell lines were cultured in polystyrene plates (Dasit, Italy).

Cell Proliferation Assay

T1 cells were plated in 96 wells-plates at a concentration of 10,000 cells/well and left to sediment for 4-5 hours. Cells were then incubated in EGCG or ECG at a concentration of 20, 40, 60, 80, 160 μg/ml for 24 and 48 hours. The substances were diluted in the culture medium starting from stock solutions (aqueous solution for EGCG and aqueous-ethanol solution (1:1) for ECG). Control cells were treated with solutions containing ethanol in amounts corresponding to that of the samples containing catechins. After 24 or 48 hours, the proliferation was calorimetrically measured using a specific kit (Cell Titer Aqueous Cell Proliferation Assay, Promega) which makes use of a colorimetric transformation reaction of colourless MTS (tetrazolium salt) into coloured formazan by living, proliferating cells. Formazan quantitation was carried out by spectrophotometric analysis at λ=490 nm.

Evaluation of Caspase 3 Activation

Caspase 3 protease activity in cells lysates was determined using a Promega kit. T1 Cells were treated with EGCG and ECG (80 μg/ml) alone or in combination with a caspase inhibitor (Z-VAD-FMK) for 24 or 48 hours. Afterwards a calorimetric substrate (DEVD-pNA) able to evidence the protease activity was added. After incubation for 4 hours at 37° C, the protease activity was determined by spectrophotometric analysis at λ=405 nm.

1. Method of preparing medicaments, dietary supplements or alimentary products with antioxidant and antitumor activities which comprises using an effective amount of an extract of Ceratonia siliqua leaves and pod containing flavonoids.

2. The method as claimed in claim 1, wherein the Ceratonia siliqua extract is a dry extract.

3. The method as claimed in claim 1, wherein the Ceratonia siliqua extract is an aqueous extract.

4. The method of an extract as claimed in claim 1, wherein flavonoids are gallic acid, (−)-epigallocatechin gallate, (−)-epicatechin gallate.

5. A process for the preparation of Ceratonia siliqua leaves and pod extracts containing flavonoids comprising the following steps:
a) infusing the leaves or pod meal in hot distilled water; b) centrifuging the aqueous extract;
c) filtering the aqueous extract;
d) evaporating off water;
e) optionally repeating steps a)-d) on the centrifugation residue from step b).

6. A process as claimed in claim 5, wherein 50 to 200 ml of water are used per 1 g of leaves or pod meal.

7. A process as claimed in claim 5, wherein 100 ml of water are used per 1 g of leaves or pod meal.

8. Extracts obtainable by the process of claim 5.

9. Pharmaceutical preparations, dietary supplements or alimentary products containing the extract of claim 8.
